(19) United States
(12) Patent Application Publication
(10) Pub. No.: US 2024/0409641 A1
(43) Pub. Date: Dec. 12, 2024

(54) DUAL ACTING CD1D IMMUNOGLOBULIN FOR TREATMENT OF MYELODYSPLASTIC SYNDROMES

(71) Applicant: LAVA THERAPEUTICS N.V., Utrecht (NL)

(72) Inventor: Johannes Jelle VAN DER VLIET, Amsterdam (NL)

(21) Appl. No.: 18/736,980

(22) Filed: Jun. 7, 2024

Related U.S. Application Data
(60) Provisional application No. 63/472,133, filed on Jun. 9, 2023.

(51) Int. Cl. C07K 16/28; A61K 38/20

(52) U.S. Cl. CPC C07K 16/2833; A61K 38/2013; C07K 16/2809; C07K 2317/31

(57) ABSTRACT

The present disclosure provides multi-specific immunoglobulins that bind to CD1d and a gamma-delta TCR, pharmaceutical preparations and use of such multi-specific binding moieties and/or immunoglobulins in the treatment of a myelodysplastic syndrome.

### CROSS-REFERENCE TO RELATED APPLICATIONS

The present application claims priority to U.S. Provisional Application 63/472,133, filed Jun. 9, 2023, the contents of which are incorporated herein by reference in its entireties.

### SEQUENCE LISTING

The contents of the electronic sequence listing (LVAT_027_01US_SeqList_ST26.xml; Size: 14,305 bytes; and Date of Creation: Jun. 7, 2024) are herein incorporated by reference in its entirety.

### BACKGROUND

CD1d is a member of the CD1 (cluster of differentiation 1) family of glycoproteins (including CD1a, CD1b, CD1c, CD1d and CD1e) and is expressed on the surface of various human cells, including antigen presenting cells (APC). They are non-classical MHC proteins, related to the class I MHC proteins, and are involved in the presentation of lipid antigens to a subgroup of T cells. In human CDld is encoded by CD1D, also known as R3G1. APCs displaying CD1d include B-cells, dendritic cells (e.g. in lymph nodes), and monocytes. CD1d is also expressed by various other cell types, for example, in liver, pancreas, skin, kidney, uterus, conjunctiva, epididymis, thymus and tonsil (Canchis et al. (1992) Immunology 80:561).

Cells that are activated/stimulated via CD1d include CD1d restricted Natural Killer T-cells (NKT cells). NKT cells are a heterogeneous group of T cells that share properties of both T cells and natural killer cells. NKT cells are a subset of T cells that express an alpha(a)/beta(3) T-cell receptor (TCR), as well a variety of molecular markers that are typically associated with NK cells.

### SUMMARY

For the reasons indicated above, modulation of CD1d-mediated effects is of potential therapeutic benefit. 1D12-5C8 VAR is a Vγ9Vδ2-T cell engaging bispecific antibody of 27.3 kDa that engages Vγ9Vδ2-T cells in the killing of tumor cells in a tumor target-dependent manner. Vγ9Vδ2-T cells are a population of T cells that have a critical role in immune surveillance with an ability to detect and target tumor cells. The Vγ9Vδ2-T cell receptors (TCRs) on these cells sense conformational changes in butyrophilin (BTN)3A1 that are induced by phosphoantigens, which are generally expressed at higher levels in tumor cells. Presence of Vγ9Vδ2-T cells in blood and solid tumors strongly correlates with patient survival supporting their importance. Approaches that improve targeting and activation of Vγ9Vδ2-T cells to tumors are thought to have outstanding potential for the development of novel, efficacious and safe treatments for cancer.

1D12-5C8 VAR consists of two VHH (single variable domain of the heavy chain of heavy-chain only antibodies) domain antibodies linked via a 5 amino acid glycine-serine linker. One arm recognizes the Vδ2 chain of the γδ-TCR and thereby targets Vγ9Vδ2-T cells, the other arm is specific for the tumor antigen cluster of differentiation (CD)1d; thus 1D12-5C8 VAR cross-links Vγ9Vδ2-T cells to tumor cells resulting in the activation of the Vγ9Vδ2-T cells. This will lead to degranulation of the Vγ9Vδ2-T cells, the secretion of cytolytic molecules and the subsequent death of the cancer cells.

In addition, 1D12-5C8 VAR is able to induce iNKT cell activation via binding to CD1d and stabilization of the interaction between CD1d and the invariant TCR of iNKT cells. Activated iNKT cells can exert direct cytotoxicity against CD1d-expressing tumor cells and, in addition, produce various cytokines that promote the activity and cytotoxic potential of other immune cells, including Vγ9Vδ2-T cells, to induce subsequent tumor cell lysis. Further, as it is known that Vγ9Vδ2-T cells can also act as antigen presenting cells, they can prime naive CD4 and CD8 T cell responses upon their activation. This unique feature may contribute to the initiation and propagation of “conventional” T cell responses, resulting in a further enhancement of the antitumor immune response.

In normal physiology, CD1d and its family members are structurally related to major histocompatibility complex (MHC) class I glycoproteins and they are involved in the presentation of lipid antigens to CD1d-restricted T cells, including iNKT cells. Based on the sponsor's analyses of patient samples, patient-derived CLL, MM and AML cells revealed that CD1d is expressed at varying mean fluorescence (MF) index levels per indication (CLL MF index 1.0-114.4, MM 0.9 to 159.2, AML 0.7 and 32.7). Vγ9Vδ2-T cell mediated tumor cell lysis was consistently observed in the majority of patient samples displaying a broad range of CD1d MF indices from ˜1 to ˜94. The unique features of 1D12-5C8 VAR and the capabilities of the unique effector cells that it targets are believed to have the potential of making a substantial beneficial impact on the treatment of patients with relapsed/refractory CLL, MM or AML. Despite current treatment options, there is still an unmet need for patients diagnosed with these diseases as the majority of patients will experience relapse of disease, are refractory to or develop resistance to therapies and will eventually succumb to the consequences of the disease.

In this trial, low dose subcutaneous (LDSC) IL-2 will be explored as an immune modulator for 1D12-5C8 VAR in separate dose-finding cohorts based on its known T-cell stimulatory effect. IL-2 has been shown to induce rapid expansion of Vγ9Vδ2-T cells in vitro and to also support Vγ9Vδ2-T cell expansion in both non-human primates and humans exposed to phosphoantigen stimulation. IL-2 therefore has the potential to expand and enhance the availability of Vγ9Vδ2-T cells in patients treated with 1D12-5C8 VAR to promote more potent responses towards tumor cells. LDSC IL-2 is currently used and studied for the management of immune mediated diseases, and well tolerated when administered at low doses (1 million international units [MIU]/day).

### DETAILED DESCRIPTION

### Definitions

The term “human Vδ2”, when used herein, refers to the rearranged δ2 chain of the Vγ9Vδ2-T cell receptor (TCR). UniProtKB—A0JD36 (A0JD36_HUMAN) gives an example of a variable TRDV2 sequence.

The term “human Vγ9”, when used herein, refers to the rearranged γ9 chain of the Vγ9Vδ2-T cell receptor (TCR). UniProtKB—Q99603_HUMAN gives an example of a variable TRGV9 sequence.

The terms “IL-2” or “IL2”, when used herein, refer to an interleukin-2 protein. UniProtKB—P60568 provides human IL-2.

The term “immunoglobulin” as used herein is intended to refer to a class of structurally related glycoproteins consisting of two pairs of polypeptide chains, one pair of light (L) chains and one pair of heavy (H) chains, all four potentially inter-connected by disulfide bonds. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. IgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Chothia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “isotype” as used herein, refers to the immunoglobulin (sub)class (for instance IgG1, IgG2, IgG3, IgG4, IgD, IgA, IgE, or IgM) or any allotype thereof, such as IgG1m(za) and IgG1m(f) that is encoded by heavy chain constant region genes. Each heavy chain isotype can be combined with either a kappa (κ) or lambda (A) light chain. An antibody of the invention can possess any isotype.

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The antigen-binding region (or antigen-binding domain) which interacts with an antigen may comprise variable regions of both the heavy and light chains of the immunoglobulin molecule or may be a single-domain antigen-binding region, e.g. a heavy chain variable region only. The constant regions of an antibody, if present, may mediate the binding of the immunoglobulin to host tissues or factors, including various cells of the immune system (such as effector cells and T cells) and components of the complement system such as C1q, the first component in the classical pathway of complement activation.

The Fc region of an immunoglobulin is defined as the fragment of an antibody which would be typically generated after digestion of an antibody with papain which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system.

The term “hinge region” as used herein is intended to refer to the hinge region of an immunoglobulin heavy chain. Thus, for example, the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

Reference to amino acid positions in the Fc region/Fc domain in the present invention is according to the EU-numbering (Edelman et al., Proc Natl Acad Sci USA. 1969 May; 63(1):78-85; Kabat et al., Sequences of proteins of immunological interest. 5th Edition—1991 NIH Publication No. 91-3242).

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′)2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise indicated by context. Although such fragments are generally included within the meaning of antibody, they collectively and each independently are unique features of the present invention, exhibiting different biological properties and utility. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies of the invention, the first antigen-binding region or the second antigen-binding region, or both, is a single domain antibody. Single domain antibodies are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single domain antibodies comprise a single CDR1, a single CDR2 and a single CDR3. Examples of single domain antibodies are variable fragments of heavy-chain-only antibodies, antibodies that naturally do not comprise light chains, single domain antibodies derived from conventional antibodies, and engineered antibodies. Single domain antibodies may be derived from any species. For example, single domain antibodies can be derived from antibodies raised in Camelidae species, for example in camel, dromedary, llama, alpaca and guanaco. Like a whole antibody, a single domain antibody is able to bind selectively to a specific antigen. Single domain antibodies may contain only the variable domain of an immunoglobulin chain, i.e. CDR1, CDR2 and CDR3 and framework regions. Such antibodies are also called Nanobody®, or VHH.

The term “parent antibody”, is to be understood as an antibody which is identical to an antibody according to the invention, but wherein the parent antibody does not have one or more of the specified mutations. A “variant” or “antibody variant” or a “variant of a parent antibody” of the present invention is an antibody molecule which comprises one or more mutations as compared to a “parent antibody”. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), |
| | Leu (L), and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | Q, T, K, S, G, N, D, E, and R |

In the context of the present invention, a substitution in a variant is indicated as: Original amino acid—position—substituted amino acid; The three-letter code, or one letter code, are used, including the codes Xaa and X to indicate amino acid residue. Accordingly, the notation “T366W” means that the variant comprises a substitution of threonine with tryptophan in the variant amino acid position corresponding to the amino acid in position 366 in the parent antibody.

Furthermore, the term “a substitution” embraces a substitution into any one of the other nineteen natural amino acids, or into other amino acids, such as non-natural amino acids. For example, a substitution of amino acid T in position 366 includes each of the following substitutions: 366A, 366C, 366D, 366G, 366H, 366F, 366I, 366K, 366L, 366M, 366N, 366P, 366Q, 366R, 366S, 366E, 366V, 366W, and 366Y.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes. Such epitopes may be on the same or on different target antigens. If the epitopes are on different targets, such targets may be on the same cell or different cells or cell types. A multispecific antibody may comprise one or more single-domain antibodies.

The term “bispecific antibody” refers to an antibody having specificities for two different, typically non-overlapping, epitopes. Such epitopes may be on the same or different targets. If the epitopes are on different targets, such targets may be on the same cell or different cells or cell types. A bispecific antibody may comprise one or two single-domain antibodies.

Examples of different classes of multispecific, such as bispecific, antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287(52): 43331-9, doi: 10.1074/jbc.M112.397869. Epub 2012 Nov. 1), DIG-body and PIG-body (Pharmabcine, WO2010134666, WO2014081202), the Strand Exchange Engineered Domain body (SEEDbody)(EMD Serono), the Biclonics (Merus, WO2013157953), FcΔAdp (Regeneron), bispecific IgG1 and IgG2 (Pfizer/Rinat), Azymetric scaffold (Zymeworks/Merck), mAb-Fv (Xencor), bivalent bispecific antibodies (Roche, WO2009080254) and DuoBody® molecules (Genmab).

Examples of recombinant IgG-like dual targeting molecules include but are not limited to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-April; 5(2):208-18), approaches with common light chain, KABodies (NovImmune, WO2012023053) and CovX-body® (CovX/Pfizer, Doppalapudi, V. R., et al 2007. Bioorg. Med. Chem. Lett. 17,501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February; 32(2):191-8), Ts2Ab (Medlmmune/AZ, Dimasi et al. J Mol Biol. 2009 Oct. 30; 393(3):672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to ScFv/Fc Fusions (Academic Institution, Pearce et al Biochem Mol Biol Int. 1997 September; 42(6):1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual(ScFv)2-Fab (National Research Center for Antibody Medicine—China).

Examples of Fab fusion bispecific antibodies include but are not limited to F(ab)2 (Medarex/AMGEN), Dual-Action or Bis-Fab (Genentech), Dock-and-Lock® (DNL) (ImmunoMedics), Bivalent Bispecific (Biotecnol) and Fab-Fv (UCB-Celltech).

Examples of ScFv-, diabody-based and domain antibodies include but are not limited to Bispecific T Cell Engager (BiTE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3; 425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88(6):667-75), dual targeting Nanobodies® (Ablynx, Hmila et al., FASEB J. 2010), dual targeting heavy chain only domain antibodies.

In some embodiments, the multispecific antibody used in the invention is in a VHH-Fc format, i.e. the antibody comprises two or more single-domain antigen-binding regions that are linked to each other via a human Fc region dimer. In this format, each single-domain antigen-binding region is fused to an Fc region polypeptide and the two fusion polypeptides form a dimeric bispecific antibody via disulfide bridges in the hinge region. Such constructs typically do not contain full, or any, CH1 or light chain sequences. FIG. 12B of WO06064136 provides an illustration of an example of this format.

In the context of antibody binding to an antigen, the terms “binds”, “capable of binding” or “specifically binds” refer to the binding of an antibody to a predetermined antigen or target (e.g. human Vδ2 or human CD1d) to which binding typically is with an apparent affinity corresponding to a KD of about 10-6 M or less, e.g. 10-7 M or less, such as about 10-8 M or less, such as about 10-9 M or less, about 10-10 M or less, or about 10-11 M or even less, e.g. when determined using flow cytometry. Alternatively, KD values can be determined using for instance surface plasmon resonance (SPR) technology in a BIAcore T200 or bio-layer interferometry (BLI) in an Octet RED96 instrument using the antigen as the ligand and the binding moiety or binding molecule as the analyte. Specific binding means that the antibody binds to the predetermined antigen with an affinity corresponding to a KD that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The degree with which the affinity is lower is dependent on the KD of the binding moiety or binding molecule, so that when the KD of the binding moiety or binding molecule is very low (that is, the binding moiety or binding molecule is highly specific), then the degree with which the affinity for the antigen is lower than the affinity for a non-specific antigen may be at least 10,000-fold. The term “KD” (M), as used herein, refers to the dissociation equilibrium constant of a particular interaction between the antigen and the binding moiety or binding molecule.

“Capable of binding the Vδ2 chain of a Vγ9Vδ2-TCR” or “binding the Vδ2 chain of a Vγ9Vδ2-TCR” or the like means that the antibody can bind the Vδ2 chain as a separate molecule and/or as part of a Vγ9Vδ2-TCR. However, the antibody will not bind to the Vγ9 chain as a separate molecule.

“Capable of binding the Vγ9 chain of a Vγ9Vδ2-TCR” or “binding the Vγ9 chain of a Vγ9Vδ2-TCR” or the like means that the antibody can bind the Vγ9 chain as a separate molecule and/or as part of a Vγ9Vδ2-TCR. However, the antibody will not bind to the Vδ2 chain as a separate molecule.

In the context of the present invention, “competition” or “able to compete” or “competes” refers to any detectably significant reduction in the propensity for a particular binding molecule (e.g. an CD1d binding antibody) to bind a particular binding partner (e.g. CD1d) in the presence of another molecule (e.g. a different CD1d antibody) that binds the binding partner. Typically, competition means an at least about 25 percent reduction, such as an at least about 50 percent, e.g. an at least about 75 percent, such as an at least 90 percent reduction in binding, caused by the presence of another molecule, such as an antibody, as determined by, e.g., ELISA analysis or flow cytometry using sufficient amounts of the two or more competing molecules, e.g. antibodies. Additional methods for determining binding specificity by competitive inhibition may be found in for instance Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley InterScience N. Y., (1992, 1993), and Muller, Meth. Enzymol. 92, 589-601 (1983)).

The method or use of the invention may involve the use of an antibody that binds to the same epitope on a target (e.g. Vδ2 or CD1d) as antibody described herein. There are several methods available for mapping antibody epitopes on target antigens known in the art, including but not limited to: crosslinking coupled mass spectrometry, allowing identification of peptides that are part of the epitope, and X-ray crystallography identifying individual residues on the antigen that form the epitope. Epitope residues can be determined as being all amino acid residues with at least one atom less than or equal to 5 Å from the antibody. 5 Å was chosen as the epitope cutoff distance to allow for atoms within a van der Waals radius plus a possible water-mediated hydrogen bond. Next, epitope residues can be determined as being all amino acid residues with at least one atom less than or equal to 8 Å. Less than or equal to 8 Å is chosen as the epitope cutoff distance to allow for the length of an extended arginine amino acid. Crosslinking coupled mass spectrometry begins by binding the antibody and the antigen with a mass labeled chemical crosslinker. Next the presence of the complex is confirmed using high mass MALDI detection. Because after crosslinking chemistry the Ab/Ag complex is extremely stable, many various enzymes and digestion conditions can be applied to the complex to provide many different overlapping peptides. Identification of these peptides is performed using high resolution mass spectrometry and MS/MS techniques. Identification of the crosslinked peptides is determined using mass tag linked to the cross-linking reagents. After MS/MS fragmentation and data analysis, peptides that are crosslinked and are derived from the antigen are part of the epitope, while peptides derived from the antibody are part of the paratope. All residues between the most N- and C-terminal crosslinked residue from the individual crosslinked peptides found are considered to be part of the epitope or paratope.

The terms “first” and “second” antigen-binding regions when used herein do not refer to their orientation/position in the antibody, i.e. they have no meaning with regard to the N- or C-terminus. The terms “first” and “second” only serve to correctly and consistently refer to the two different antigen-binding regions in the claims and the description.

“% sequence identity”, when used herein, refers to the number of identical nucleotide or amino acid positions shared by different sequences (i.e., % identity=# of identical positions/total # of positions×100), taking into account the number of gaps, and the length of each gap, which need to be introduced for optimal alignment. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4.

“Treatment” or “treating” refers to the administration of an effective amount of an antibody and/or dose of IL-2 according to the present invention with the purpose of easing, ameliorating, arresting, eradicating (curing) or preventing symptoms or disease states. An “effective amount” refers to an amount effective, at dosages and for periods of time necessary, to achieve a desired therapeutic result. An effective amount of a polypeptide, such as an antibody, may vary according to factors such as the disease stage, age, sex, and weight of the individual, and the ability of the antibody to elicit a desired response in the individual. An effective amount is also one in which any toxic or detrimental effects of the antibody are outweighed by the therapeutically beneficial effects. Administration may be carried out by any suitable route, but will typically be parenteral, such as intravenous, intramuscular or subcutaneous.

As described above, in some embodiments the invention relates to a method for the treatment of myelodysplastic syndrome comprising administering to a subject in need thereof, of: (i) a multispecific antibody comprising a first antigen-binding region capable of binding CD1d and a second antigen-binding region capable of binding a human Vγ9Vδ2 T cell receptor. In some embodiments, the method further comprises administering a low-dose of IL-2 of less than 2 MIU/day.

Suitable formulations for multispecific antibodies capable of binding human CD1d and capable of binding the Vδ2 chain of a human Vγ9Vδ2 T cell receptor for use in the invention have been described. The antibody may be formulated at a strength of 1 mg/mL in 10 mM histidine, 1 mM methionine, 280 mM sucrose 0.02%, polysorbate 80, pH 6.0. The multispecific antibody may be administered via any suitable administration route, for example intravenously or subcutaneously.

The IL-2 used in the method of the invention may be recombinant IL-2. IL-2 may be Aldesleukin (Proleukin©) for example presented in a 5 mL vial with 1.3 mg Aldesleukin/Proleukin (22×10⁶ IU (international units) per vial), wherein the product can be reconstituted using sterile water for injection. Other suitable IL-2 analogs include NKTR-214 (Bempegaldesleukin/Nektar), ALK 4230 (nemvaleukin/Aalkermes-Reliant), SAR444245—THOR 707/Synthorx/Sanofi) and XTX-202 (Xilio). IL-2 may be administered via any suitable administration route, for example subcutaneously.

Amino acid sequences of the multispecific antibodies are provided below.

| SEQ | | | |
|---|---|---|---|
| ID. | code | Description | Sequence |
| 1 | 5C8 | CDR1 | NYAMG |
| 2 | 5C8 var1 | CDR1 | NYAMS |
| 3 | 5C8 | CDR2 | AISWSGGSTSYADSVKG |
| 4 | 5C8 | CDR3 | QFSGADYGFGRLGIRGYEYDY |
| 5 | 5C8 | VHH | EVQLVESGGGLVQAGGSLRLSCAASGRPFSNYAMGWFRQAP |
| | | | GKEREFVAAISWSGGSTSYADSVKGRFTISRDNAKNTVYLQ |
| | | | MNSPKPEDTAIYYCAAQFSGADYGFGRLGIRGYEYDYWGQG |
| | | | TQVTVSS |
| 6 | 5C8 | VHH | EVQLLESGGGSVQPGGSLRLSCAASGRPFSNYAMSWFRQAP |
| | var1 | | GKEREFVSAISWSGGSTSYADSVKGRFTISRDNSKNTLYLQ |
| | | | MNSLRAEDTAVYYCAAQFSGADYGFGRLGIRGYEYDYWGQG |
| | | | TQVTVSS |
| 7 | 5C8 | VHH | EVQLLESGGGLVQPGGSLRLSCAASGRPFSNYAMSWFRQAP |
| | var2 | | GKEREFVSAISWSGGSTSYADSVKGRFTISRDNSKNTLYLQ |
| | | | MNSLRAEDTAVYYCAAQFSGADYGFGRLGIRGYEYDYWGQG |
| | | | TLVTVSS |
| 8 | 1D12 | CDR1 | DNVMG |
| 9 | 1D12 | CDR2 | TIRTGGSTNYADSVKG |
| 10 | 1D12 | CDR3 | TIPVPSTPYDY |
| 11 | 1D12 | VHH | QVQLVESGGGLVQAGGSLRLSCAASGSMFSDNVMGWYRQAP |
| | | | GKQRELVATIRTGGSTNYADSVKGRFTISRDNAKNTVYLQM |
| | | | NSLKPEDTAVYYCRHTIPVPSTPYDYWGQGTQVTVSS |
| 12 | 1D12 var | VHH | EVQLVESGGGLVQAGGSLRLSCAASGSMFSDNVMGWYRQAP |
| | | | GKQRELVATIRTGGSTNYADSVKGRFTISRDNAKNTVYLQM |
| | | | NSLKPEDTAVYYCRHTIPVPSTPYDYWGQGTQVTVSS |
| 13 | 1D12- | Bispecific | QVQLVESGGGLVQAGGSLRLSCAASGSMFSDNVMGWYRQAP |
| | 5C8 | binding | GKQRELVATIRTGGSTNYADSVKGRFTISRDNAKNTVYLQM |
| | | molecule | NSLKPEDTAVYYCRHTIPVPSTPYDYWGQGTQVTVSSGGGG |
| | | | SEVQLVESGGGLVQAGGSLRLSCAASGRPFSNYAMGWFRQA |
| | | | PGKEREFVAAISWSGGSTSYADSVKGRFTISRDNAKNTVYL |
| | | | QMNSPKPEDTAIYYCAAQFSGADYGFGRLGIRGYEYDYWGQ |
| | | | GTQVTVSS |
| 14 | 1D12- | Bispecific | EVQLVESGGGLVQAGGSLRLSCAASGSMFSDNVMGWYRQAP |
| | 5C8 var | binding | GKQRELVATIRTGGSTNYADSVKGRFTISRDNAKNTVYLQM |
| | | molecule | NSLKPEDTAVYYCRHTIPVPSTPYDYWGQGTQVTVSSGGGG |
| | | | SEVQLLESGGGSVQPGGSLRLSCAASGRPFSNYAMSWFRQA |
| | | | PGKEREFVSAISWSGGSTSYADSVKGRFTISRDNSKNTLYL |
| | | | QMNSLRAEDTAVYYCAAQFSGADYGFGRLGIRGYEYDYWGQ |
| | | | GTQVTVSS |

The multispecific antibody may be administered with any suitable dosing interval, for example a dosing interval of 1 day to 1 month, for example a dosing interval of 1 to 21 days, such as 2, 3, 4, 5, 6, 7, 8, 9, 10, 11, 12, 13, 14, 15, 16, 17, 18, 19, 20 or 21 days or a dose interval of between 7 and 21 days, such as 7 or 14 days.

IL-2 may be administered with any suitable dosing interval, for example a dosing interval of 1 day to 1 month, for example a dosing interval of 1 to 21 days, such as 1, 2, 3, 4, 5, 6, 7, 8, 9, 10, 11, 12, 13, 14, 15, 16, 17, 18, 19, 20 or 21 days or a dose interval of between 7 and 21 days, such as 7 or 14 days.

IL-2 may be administrated before, on the same day, or after the multispecific antibody. For example, IL-2 may be administered daily for at least 1 day, such as 2, 3, 4, 5, 6, 7 or more days after administration of the multispecific antibody, for example starting the day after administration of the multispecific antibody.

For example, the multispecific antibody may be administered on day 1 and IL-2 may be administered on day 2 or on day 2, 3 and 4. Such a cycle may be repeated at least 1 time, such as 2, 3, 4, 5, 6, 7, 8 or more times, e.g. with intervals between the multispecific antibody administration of 14 days.

Thus, for example, the multispecific antibody may be administered as intravenous infusion with a 14-day dosing interval and IL-2 as a single dose or as 3 daily doses on three consecutive days starting at 24 hours after the start of the multispecific antibody infusion, for a total duration of 4 cycles.

The multispecific antibody may be administered at any suitable dose, for example a dose of at least 20 micrograms per administration, for example at least 40 micrograms, such as at least 80 micrograms, for example at least 120 micrograms, such as at least 240 micrograms, for example at least 360 micrograms, such as at least 500 micrograms, such as at least 1000 micrograms, for example at least 3 mg, for example at least 6 mg per administration or at a dose between 360 micrograms and 10 mg per administration.

For example, the multispecific antibody may be administered once every 14 days at a dose of at least 20 micrograms, for example at least 40 micrograms, such as at least 80 micrograms, for example at least 120 micrograms, such as at least 240 micrograms, for example at least 360 micrograms, such as at least 500 micrograms, such as at least 1000 micrograms, for example at least 3 mg, for example at least 6 mg or at a dose between 360 micrograms and 10 mg.

Alternatively, the multispecific antibody may be administered twice every 14 days at a dose of at least 20 micrograms per administration, for example at least 40 micrograms, such as at least 80 micrograms, for example at least 120 micrograms, such as at least 240 micrograms, for example at least 360 micrograms, such as at least 500 micrograms, such as at least 1000 micrograms, for example at least 3 mg, for example at least 6 mg per administration or at a dose between 360 micrograms and 10 mg per administration.

The multispecific antibody may be administered at a dose of at least 20 micrograms, for example at least 0.5 micrograms/kg, such as at least 1 microgram/kg, for example at least 2 micrograms/kg, such as at least 240 micrograms/kg, for example at least 360 micrograms/kg, such as at least 500 micrograms, such as at least 1000 micrograms/kg, for example at least 3 mg/kg, for example at least 6 mg/kg or at a dose between 360 micrograms/kg and 10 mg/kg.

For example, the multispecific antibody may be administered once per 14 days at a dose of at least 20 micrograms, for example at least 0.5 micrograms/kg, such as at least 1 microgram/kg, for example at least 2 micrograms/kg, such as at least 240 micrograms/kg, for example at least 360 micrograms/kg, such as at least 500 micrograms, such as at least 1000 micrograms/kg, for example at least 3 mg/kg, for example at least 6 mg/kg or at a dose between 360 micrograms/kg and 10 mg/kg.

IL-2 may be administered at any suitable dose, such as any suitable daily dose. IL-2 may for example be given at a daily dose of between 0.1 and 5 MIU (0.1 and 5 10⁶ IU), such as a dose of between 0.2 and 2 MIU/day, such as between 0.5 and 1.5 MIU/day, e.g. 0.5, 0.6, 0.7, 0.8, 0.9, 1, 1.1, 1.2, 1.3, 1.4 or 1.5 MIU/day. IL-2 may also be given twice daily, for example twice daily at a dose of between 0.1 and 5 MIU (0.1 and 5 10⁶ IU), such as a dose of between 0.2 and 2 MIU/day, such as between 0.5 and 1.5 MIU, e.g. 0.5, 0.6, 0.7, 0.8, 0.9, 1, 1.1, 1.2, 1.3, 1.4 or 1.5 MIU.

Myelodysplastic syndromes (MDS) are clonal haematopoietic neoplasms defined by cytopenias and morphologic dysplasia. MDS have an annual age-adjusted incidence rate of approximately 4.4 to 4.6 cases per 100,000 people. Prognosis is directly related to the number of bone marrow blast cells, to certain cytogenetic abnormalities, and to the amount of peripheral blood cytopenias.

MDS occur predominantly in adults with median age at diagnosis of approximately 70 years. Anemia, bleeding, easy bruising, and fatigue are common initial findings. Approximately 50% of patients have a detectable cytogenetic abnormality, most commonly a deletion of all or part of chromosome 5 or 7, or trisomy 8.

A variety of pathological and risk classification systems have been developed to predict the overall survival of patients with MDS and the evolution from MDS to AML. Major prognostic classification systems include the International Prognostic Scoring System (IPSS); the WHO Prognostic Scoring System (WPSS); and the MD Anderson Cancer Center Prognostic Scoring Systems. Clinical variables in these systems have included bone marrow and blood myeloblast percentage, specific cytopenias, transfusion requirements, age, performance status, and bone marrow cytogenetic abnormalities.

High risk patients, besides some specific subtypes of MDS, and patients who do not respond or have ceased responding to DNA methyltransferase inhibitors have limited treatment options and should be considered for enrollment in clinical trials.

### Kits

In a further aspect, the invention relates to a kit for the treatment of Myelodysplastic syndromes comprising: a multispecific antibody comprising a first antigen-binding region capable of binding human CD1d and a second antigen-binding region capable of binding a human Vγ9Vδ2 T cell receptor. In some embodiments, the kit further comprises a dose, such as a daily dose, of IL-2 less than 2 MIU, optionally comprising instructions for use.

The kit may further comprise a solvent for the dilution of the multispecific antibody the dose of IL-2.

Antibodies may be formulated with pharmaceutically-acceptable excipients in accordance with conventional techniques such as those disclosed in Rowe et al. 2012 Handbook of Pharmaceutical Excipients, ISBN 9780857110275). The pharmaceutically-acceptable excipient as well as any other carriers, diluents or adjuvants should be suitable for the antibodies and the chosen mode of administration. Suitability for excipients and other components of pharmaceutical compositions is determined based on the lack of significant negative impact on the desired biological properties of the chosen antibody or pharmaceutical composition of the present invention (e.g., less than a substantial impact (10% or less relative inhibition, 5% or less relative inhibition, etc.) upon antigen binding). A pharmaceutical composition may include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition. Further pharmaceutically-acceptable excipients include any and all suitable solvents, dispersion media, coatings, antibacterial and antifungal agents, isotonicity agents, antioxidants and absorption-delaying agents and the like that are physiologically compatible with an antibody of the present invention.

### EXAMPLES

### Example 1

This is an open-label, multi-center, Phase 1 dose escalation and Phase 2a dose expansion trial to investigate the safety, tolerability, pharmacokinetics, pharmacodynamics, immunogenicity and antitumor activity of 1D12-5C8 VAR in relapsed or refractory CLL, MM or AML patients. The trial consists of two parts. The first is a Phase 1 open-label, dose escalation part to determine the safety and recommended dose of 1D12-5C8 var (±LDSC IL-2) for Phase 2a (RP2D). The second is a Phase 2a open-label dose expansion part, in which the number of patients will be expanded in one or more disease specific cohorts (CLL, MM or AML) to confirm safety and assess preliminary antitumor activity (per disease cohort) of the respective recommended dose and regimen established in the first part of the trial.

| | |
|---|---|
| IMP Name | 1D12-5C8 var |
| Type | Biologic |
| Dose Formulation | 10 mM Histidine, 50 mM Glycine, 10 mM Methionine, 6% |
| | Trehalose, 0.01% Polysorbate 80, pH 6.5 |
| Unit Dose | 1 mg/mL |
| Strength(s) | |
| Dose regimen | Part 1 IV administration: 2-hour infusion at Day 1, 8, 11, 15, 18, 22, 25 in |
| | the first treatment cycle of 28 days and twice weekly thereafter |
| | IV administration will occur in cohort 1-11. In cohort 5 and cohorts A, one |
| | of the doses will be administered by SC injection (for cohort 5 one SC |
| | dose at Day 8 of Cycle 1 and for cohorts A one SC dose at Day 1 of Cycle 2). |
| | Part 1 SC administration: Injection at Day 1, 8, 11, 15, 18, 22, 25 in the first |
| | treatment cycle of 28 days and twice weekly thereafter. |
| | SC administration will occur in cohorts C (and D) and AML3. |
| | Part 2: 2-hour infusion or SC administration twice weekly or a different |
| | regimen as established in Part 1 of the trial. |
| Route of | IV infusion. IMP IV infusion is to be administered under the supervision of |
| Administration | authorized site staff for a duration of 2-hours +/−15 minutes. It must not be |
| | administered as a bolus. Refer to the Pharmacy Manual including IMP |
| | handling instructions. |
| | SC injection. IMP SC injection is to be administered under the supervision |
| | of authorized site staff. Refer to the Pharmacy Manual including |
| | IMP handling instructions. |
| | The preferred injection site will be the abdomen*; the upper arm and thigh |
| | are considered as acceptable alternative sites. Injection sites may be |
| | rotated for consecutive SC doses, if needed. |
| Use | Experimental |
| Sourcing | Provided centrally by the sponsor |
| Packaging and | IMP will be provided in 2R DIN vial. Each 2R DIN vial will be labeled as |
| Labeling | required per country requirement. Additional Diluent (10 mM Histidine, |
| | 50 mM Glycine, 10 mM Methionine, 6% Trehalose, 5% Polysorbate |
| | 80, pH 6.5) will be in 10R DIN vials. Each Diluent vial will be |
| | labelled as required per country requirement. |
| Additional | IL-2 |
| treatment | |
| Type | Biological |
| Dose Formulation | 5 mL vial with 1.3 mg Aldesleukin/Proleukin (22 MIU per vial). The |
| | product can be reconstituted using sterile water for injection |
| | (SWFI) |
| Dose regimen | SC IL-2 administration of 1 MIU by: |
| | Single IL-2 administration of 1 MIU at 24 hours (+/−2 hours time |
| | window) after LAVA-051 administration, weekly in Cycle 1 and |
| | Cycle 2 (Day 2, 9, 16 and 23). |
| Route of | Subcutaneous |
| Administration | |
| Use | Experimental |
| Sourcing | Provided centrally by the sponsor |
| Packaging and | IL-2 will be provided in 5 mL vial filled with 1.3 mg. Each vial will |
| Labeling | be labeled per country requirements |

*For cohort 5 (one SC doseat Day 8 of Cycle 1) and cohorts A (one SC dose at Day 1 of Cycle 2), the SC the injection site should be the abdomen.

### Phase 1 (Dose Escalation)—Part 1

### Dose Cohorts and Dosing Regimen

In the present trial, up to 11 cohorts at escalating intravenous (IV) dose levels of 1D12-5C8 VAR monotherapy are envisaged with relapsed/refractory CLL or MM. As AML is a rapidly progressing disease, patients with AML, including MDS, will be enrolled in separate parallel cohorts only from dose level 6 (or highest cleared dose) of 1D12-5C8 VAR monotherapy onwards, which represents a safe and pharmacologically active dose for enrolled patients with CLL or MM.

In addition, from cohort dose level 6 (or the highest cleared dose level) monotherapy onwards, separate parallel cohorts B will exist to evaluate the effect of (LDSC) IL 2 administered weekly, at 24 hours after 1D12-5C8 VAR infusion, for two cycles. Eligible patients with relapsed/refractory CLL or MM will receive sequentially higher doses of 1D12-5C8 VAR in subsequent dose cohorts and will receive 1D12-5C8 VAR (±LDSC IL 2) until disease progression, unacceptable toxicity, withdrawal of consent or otherwise as specified in the investigational medicinal product (IMP) discontinuation criteria with a planned treatment duration of 24 weeks. Dose escalation will continue until the RP2D(s) can be defined. Pharmacokinetic bioavailability data for SC dosing of 1D12-5C8 VAR as compared to IV will be collected during dose escalation within monotherapy cohorts (cohorts A), with initiation of separate cohort(s) evaluating SC dosing once an IV RP2D is determined.

Additional cohorts may be initiated based on the safety and tolerability, pharmacokinetic and pharmacodynamic data of enrolled cohorts to investigate e.g. intermediate or higher doses, and based on the recommendation of the Dose Escalation Committee (DEC). In order to appropriately define the optimal biological active dose, the DEC can recommend to expand the number of patients at a given dose level. Also, if tolerance of 1D12-5C8 VAR appears to be different between patients with CLL from those with MM, the DEC may recommend to separate dose escalation cohorts by disease type. The DEC consists of minimally the medical monitor, a representative of the sponsor and one of the principal investigators.

Patients will receive 1D12-5C8 VAR as a 2-hour IV infusion at the same dose level at Day 1, 8, 11, 15, 18, 22, 25 in the first treatment cycle of 28 days (Cycle 1; defined as the Dose-Limiting-Toxicity (DLT) period) and twice weekly thereafter consecutively without interruption, except when necessary to manage adverse events (AEs) with a planned treatment duration of 24 weeks (up to 6 cycles); if a patient has clinical benefit after 6 cycles, treatment may be continued after discussion with the sponsor. The 7-day evaluation period following administration on Day 1 Cycle 1 will allow for a safety (AEs and vital signs) evaluation before dosing the second dose. Grade ≥2 or higher AEs will serve as criteria for consideration to postpone the second dose (Day 8, Cycle 1). Patients in selected cohorts with 1D12-5C8 VAR monotherapy (cohort 5, cohorts 7A-11A and cohorts AML6A-AML11A, see Table 1) will receive one of the 1D12-5C8 VAR doses by SC administration in order to evaluate the bioavailability of the SC administration in patients as compared to IV administration; all other 1D12-5C8 VAR doses in these cohorts will be administered IV (up to 6 cycles).

Following the DEC evaluation of cohort 6Adose level 6 (or the highest cleared dose level) of 1D12-5C8 VAR monotherapy, separate parallel escalating cohorts B (see Table 1) will be initiated to investigate the effect of LDSC IL-2 (1 MIU). LDSC IL-2 will be administered once a week (at Day 2, 9, 16 and 23) 24 hours after end of infusion (EoI) of 1D12-5C8 VAR during the first 2 cycles of 1D12-5C8 VAR treatment (Cycle 1 and 2).

It is foreseen that single patients will be enrolled for the first 3 dose cohorts unless this single patient experiences a Grade ≥1 cytokine release syndrome (CRS) (American Society for Transplantation and Cellular Therapy [ASTCT]) or ≥ Grade 2 AE (Common Terminology Criteria and grading for Adverse Events [CTCAE]) (except if AE is clearly due to extraneous causes) during the first treatment cycle of 28 days, whereupon an additional 2 patients will be enrolled at that dose level.

From the fourth cohort onwards, or earlier if events as described above were observed, at least 3 patients will be enrolled at this dose level and at all subsequent dose cohorts until one patient develops a DLT as defined in the protocol. This will require that an additional 3 patients are enrolled into that same dose cohort.

From cohort 6 onwards, the maximum dose increment including step-doses will be limited to 50% of the prior dosing cohort.

Development of DLTs in more than 1 of 6 patients in a specific dose cohort suggests that the maximum tolerated dose (MTD) has been exceeded, and further dose escalation is not pursued. The MTD is defined at the next lower dose of LAVA 051 where ≤1 DLT occurred. Additional cohorts may be included to explore doses between the non tolerated dose and the preceding lower dose, where ≤1 DLT occurred, to more precisely define the MTD. A staggered enrollment will be applied. Within a cohort with more than 1 patient, an assessment that the second dose can be administered safely (after the 7-day evaluation period following Day 1 Cycle 1) to the first patient is needed, before subsequent patients can start at the same dose level. Enrollment in a cohort with a higher dose can only start after completion and evaluation by the DEC of the safety as well as pharmacokinetic and pharmacodynamic data (as available) of all treated patients of the previous cohort.

### Dose Escalation and RP2D(s) Determination

The planned IV and SC dose schedule for Part 1 of the trial is presented in Table I below (and Section 2.2 for the flow chart of the trial design). The table shows escalating dose levels of IV 1D12-5C8 VAR monotherapy (as represented in cohorts 1-5, 6A-11A, AML6A-AML11A). In cohort 5 and selected IV cohorts A, a SC bioavailability assessment is included by administering one of the doses of LAVA 051 by SC injection. Additional parallel cohorts B will be initiated to investigate the effect of LDSC IL 2 administration. After the determination of the IV RP2D(s) and the SC bioavailability of 1012-5C8 VAR in MM and CLL patients, one or two cohort(s) (two cohorts in case of different RP2Ds for CLL and MM) are planned with a SC administration schedule of 1012-508 VAR with or without LDSC IL-2 in MM and CLL patients. Similarly, one cohort with AML patients with a SC administration schedule of LAVA 051 with or without LDSC IL-2 is planned after the IV RP21D of AML and the SC bioavailability of 10D12-508 VAR in AML is established.

TABLE 1
Planned IV and SC dose schedule (±LDSC IL-2) for Part 1

| | | | | | Tentative IV | | |
|---|---|---|---|---|---|---|---|
| | | | | | dose levels of | | |
| | | | | | 1D12-5C8 | Low dose SC | Status at time |
| IV cohorts | | IV cohorts | | N per | VAR | IL-2 | of amendment |
| CLL, MM | | AML^{g} | | cohort | (μg/patient)^{a} | (1 MIU) | 8 (2023) |
| 1 | CLL, MM | | | 1^{b} | 0.45 | | completed |
| 2 | CLL, MM | | | 1^{b} | 3 | | completed |
| 3 | CLL, MM | | | 1^{b} | 15 | | completed |
| 4^{c} | CLL, MM | | | 3 + 3 | 45 | | completed |
| 5^{d} | CLL, MM | | | 3 + 3 | 100 | | completed |
| 6A^{e} | CLL, MM | AML6A | AML | 3 + 3 | 200 | | Enrollment |
| | | | | | | | Ongoing for |
| | | | | | | | CLL, MM |
| 6B^{f} | CLL, MM | AML6B | AML | 3 + 3 | 200 | LDSC IL-2 | |
| 7A^{e} | CLL, MM | AML7A | AML | 3 + 3 | 300 | | |
| 7B^{f} | CLL, MM | AML7B | AML | 3 + 3 | 300 | LDSC IL-2 | |
| 8A^{e} | CLL, MM | AML8A | AML | 3 + 3 | 450 | | |
| 8B^{f} | CLL, MM | AML8B | AML | 3 + 3 | 450 | LDSC IL-2 | |
| 9A^{e} | CLL, MM | AML9A | AML | 3 + 3 | 670 | | |
| 9B^{f} | CLL, MM | AML9B | AML | 3 + 3 | 670 | LDSC IL-2 | |
| 10A^{e} | CLL, MM | AML10A | AML | 3 + 3 | 1,000 | | |
| 10B^{f} | CLL, MM | AML10B | AML | 3 + 3 | 1,000 | LDSC IL-2 | |
| 11A^{e} | CLL, MM | AML11A | AML | 3 + 3 | 1,500 | | |
| 11B^{f} | CLL, MM | AML11B | AML | 3 + 3 | 1500 | LDSC IL-2 | |

| | | | | | Tentative | |
|---|---|---|---|---|---|---|
| | | | | | SC dose | |
| | | | | | levels of | Low dose SC |
| SC cohorts | | SC cohorts | | N per | 1D12-5C8 | IL-2 |
| CLL, MM | | AML | | cohort | VAR | (1 MIU) |
| C | CLL, MM | AMLC | AML | 3 + 3 | Exposure | with or |
| (and D)^{h} | | | | | equal to IV | without |
| | | | | | RP2D | LDSC IL-2 |

^{a}Subsequent dose increase/dose level will be determined based on the available safety-, pharmacokinetic-, and pharmacodynamic-data by the DEC.
^{b}Single patient cohorts, unless 1 patient experiences a Grade ≥1 CRS (ASTCT) or ≥Grade 2 AE (CTCAE) (except if AE is clearly due to extraneous causes) in the first treatment cycle of 28 days.
^{c}From cohort 4 onwards, standard 3 + 3 design.
^{d}In cohort 5, the second dose of 1D12-5C8 VAR was administered SC to evaluate bioavailability. The first dose, and other doses will bewere administered IV. PK profiles of both routes of administration serve as a basis for SC dose selection
^{e}In cohorts 7A-11A and AML6A-AML11A with 1D12-5C8 VAR monotherapy, the first dose of Cycle 2 (Cycle 2 Day 1) will be administered SC; all other doses will be administered IV. PK profiles of both routes of administration serve as a basis for SC dose selection.
^{f}Following the DEC evaluation of cohort 6Adose level 6 (or the highest cleared dose level) of 1D12-5C8 VAR monotherapy, separate parallel escalating cohorts B will be initiated to investigate the effect of LDSC IL-2 (1 MIU). Based on initial data with LDSC IL-2 at dose level 6 in cohort 6B (or the highest cleared dose level), it will be decided to further investigate the effect of LDSC IL-2 (1 MIU) at higher dose levels.
^{g}The decision to enroll AML patients will only be taken once a pharmacologically active dose for CLL and/or MM has been identified by IV administration (with or without LDSC IL-2). AMLwill be enrolled in separate parallel cohorts from dose level 6 (or highest cleared dose) of 1D12-5C8 VAR monotherapy onwards, which represents a safe and pharmacologically active dose for enrolled patients with CLL or MM.
^{h}One cohort C with CLL and MM patients in case CLL and MM have the same RP2D. Two cohorts (i.e. one cohort C for CLL patients and one cohort D for MM patients) in case of different RP2Ds.

For cohort 4 (dose level 45 μg) and subsequent cohorts, at least one patient with MM and at least one patient with CLL will be enrolled at each dose level, to allow disease-specific assessment prior to escalation to the next higher dose level for the respective patient population. Thus, it is possible that for one patient population (e.g. CLL) the dose-escalation for subsequent cohorts may happen later, compared to the other patient population (e.g. MM). Dose escalation decisions for IV cohorts will be based on the respective safety as well as pharmacokinetic and pharmacodynamic data obtained during Cycle 1 (as available) of all patients for the respective patient population treated in the previous dose cohort(s) and as reviewed regularly by the DEC.

Patients in Part 1, who receive less than 5 out of the 7 planned doses during Cycle 1 for reasons other than DLT, may not be evaluable for DLT and may be replaced with a new patient in consultation between the investigator and sponsor. A patient may also be deemed not evaluable during the DLT period based on the DEC assessment.

Dose escalation steps may be smaller and deviate from those foreseen in the above table if (i) one or more patients experience(s) a ≥ Grade 2 CRS (ASCTC) or if (ii) two or more patients experience the same type of AE if related and clinically relevant and of severity Grade ≥2 (CTCAE) or if (iii) one patient experiences a DLT at a given dose level.

The final recommendation as to the next appropriate dose level will be made by the DEC.

Dose escalation will continue until the RP2D(s) can be defined by an optimal biological active dose of LAVA 051 or until an MTD has been determined, whichever has occurred first. An optimal biological active dose is defined as a safe dose that demonstrates the greatest pharmacological activity. Binding of 1D12-5C8 VAR to peripheral blood Vγ9Vδ2-T cells (i.e. Vγ9Vδ2-TCR occupancy), changes in the activation status of Vγ9Vδ2-T cells, and changes in the frequency of Vγ9Vδ2-T cells are at present considered as the major pharmacological parameters/determinants that can, in combination with the pharmacokinetic assessment of 1D12-5C8 VAR, instruct identification of the optimal biological active dose. As for all pharmacodynamic parameters, the pattern of change of the above 3 parameters will be determined in all patients over the subsequently increasing dose levels that will be evaluated in Part 1 of the trial. Observation of a ‘flattening of a dose-response relationship’ and/or reaching a ‘maximum’ will be indicative of having reached the optimal biological active dose. For the final RP2D(s) determination, safety (including AEs that may have occurred during later cycles), early signs of antitumor activity, pharmacokinetic and pharmacodynamic data will be considered.

The DEC will make recommendations on the progress of Part 1 of this trial. The DEC will meet at least prior to all dose escalations; for 3-patient cohorts at least 1 patient should have follow up past Dose 1 Cycle 1 for 28 days, the others for 14 days; for 6-patient cohorts at least 4 patients should have follow up for 28 days, the others for 14 days. The following recommendations will be considered by the DEC based on the review of all safety-, pharmacokinetic-, pharmacodynamic- and antitumor data (as available):
(a) Replace patients not evaluable during the DLT evaluation period.
(b) Continue with the next higher dose level (±LDSC IL-2), decide on the recommended dose level and cohort size for this next dose level.
(c) Continue with the same dose level (±LDSC IL-2) as evaluated currently and add additional patients.
(d) Continue with an intermediate dose level (±LDSC IL-2) between the current dose level and the next higher dose level (implemented via protocol amendment).
(e) Continue with a dose level lower than the dose level (±LDSC IL-2) currently under evaluation.
(f) Continue with a higher dose level (±LDSC IL-2) than planned in case of no safety concerns and pharmacokinetic data of preceding dose level(s) indicating no- or limited exposure and no- or limited increase of pharmacodynamic markers is observed (implemented via protocol amendment).
(g) Continue with a different dosing regimen (e.g. frequency of administration, step-dosing etc) including adjustment of duration of infusion and route of administration (e.g. SC).
(h) Decision on/whether there is a DLT event.
(i) Decision to continue evaluating MM patients separately from CLL patients for determining RP2D of 1D12-5C8 VAR (±LDSC IL-2).
(j) Recommendation to expand a cohort at a given dose level for further characterization of specific pharmacodynamic parameters or when the dose appears to satisfy criteria for an optimal biological active dose and regimen (dose optimization).
(k) Initiating (a) cohort(s) of AML patients, once a pharmacologically active dose is determined.
(l) Determination of a dose for SC administration cohorts (with or without LDSC IL-2) based on continued bioavailability of 1D12-5C8 VAR as determined in selected cohorts and IV RP2D(s).

### Example 2: Phase 2a Dose Expansion Phase—Part 2

The dose escalation part of the trial (Part 1) will be considered to decide on the disease specific cohort(s) to confirm safety and evaluate preliminary antitumor activity in Part 2 (the Phase 2a dose expansion part) of the trial. Each disease specific expansion cohort can be initiated once sufficient data to select the RP2D(s) of 1D12-5C8 VAR in Part 1 of the trial is gathered following submission and evaluation of Part 1 data to applicable regulatory authorities. In case RP2D should differ per patient population (disease), Part 2 of the respective disease for which the RP2D is determined may start while dose selection for another disease is still ongoing in Part 1. More than one dose and regimen may be evaluated in the second part of this trial as each disease may have a different dose and regimen based on Part 1 results. in order to appropriately determine a recommended dose and regimen for further evaluation.

Patients will receive 1D12-5C8 VAR (with or without LDSC IL-2) at a dose and regimen as established in Part 1 of the trial until disease progression, unacceptable toxicity, withdrawal of consent or otherwise as specified in the IMP discontinuation criteria with a planned treatment duration of 24 weeks. Drug administrations will occur consecutively without interruption except when necessary to manage AEs.

### Primary Objectives

### Part 1 Dose Escalation

(a) To investigate the safety and tolerability of 1D12-5C8 VAR in patients with relapsed/refractory CLL, MM or AML.
(b) To determine the RP2D of 1D12-5C8 VAR in patients with relapsed/refractory CLL, MM or AML.

### Part 2 Dose Expansion

(a) To confirm the safety and tolerability of 1D12-5C8 VAR in disease specific dose expansion cohorts.

### Secondary Objectives

### Part 1 Dose Escalation and Part 2 Dose Expansion

(a) To explore the preliminary antitumor activity of 1D12-5C8 VAR.
(b) To evaluate the pharmacokinetics of 1D12-5C8 VAR.
(c) To evaluate the immunogenicity of 1D12-5C8 VAR.

### Exploratory Objectives

### Part 1 Dose Escalation and Part 2 Dose Expansion

(a) Progression free survival (PFS) and duration of response (DOR).
(b) To evaluate the pharmacodynamics of 1D12-5C8 VAR.
(c) Minimal/measurable residual disease (MRD) (only for CLL and AML patients).

Part 1 and Part 2 Receiving 1D12-5C8 VAR and Weekly LDSC IL-2 for Two Cycles
(a) To investigate the safety and tolerability of 1D12-5C8 VAR with LDSC IL-2 in patients with relapsed/refractory CLL, MM or AML.
(b) To explore the preliminary pharmacokinetics of 1D12-5C8 VAR with LDSC IL-2.
(c) To explore the preliminary pharmacodynamics of 1D12-5C8 VAR with LDSC IL-2.
(d) To explore the preliminary immunogenicity of 1D12-5C8 VAR with LDSC IL-2.
(e) To explore the preliminary antitumor activity of 1D12-5C8 VAR with LDSC IL-2.

### Primary Endpoints

### Part 1 Dose Escalation

(a) Frequency and severity of AEs using the CTCAE version 5.0 and ASTCT grading for CRS.
(b) Frequency and type of DLT.

### Part 2 Dose Expansion

(c) Frequency and severity applying CTCAE and ASTCT grading of AEs at the respective RP2D.

### Secondary Endpoints

### Part 1 Dose Escalation and Part 2 Dose Expansion

(a) Antitumor Response:
(b) For CLL patients: Response according to the most recent International Workshop on Chronic Lymphocytic Leukemia (iwCLL) guideline.
(c) For MM patients: Response according to the most recent International Myeloma Working Group (IMWG) criteria.
(d) For AML patients: Response according to the most recent European LeukemiaNet (ELN) criteria.

### Pharmacokinetic Parameters

(a) Presence or development of anti-1D12-5C8 VAR antibodies.

### Exploratory Endpoints

### Part 1 Dose Escalation and Part 2 Dose Expansion

(a) PFS and DOR.

### Pharmacodynamic Markers.

(a) MRD (only for CLL and AML patients if clinically indicated).

### Patient Population

Approximately 5484 patients will be included in Part 1 of this trial assuming up to 11 cohorts with escalating IV dose levels (and up to 6 parallel IV dose levels+LDSC IL-2) including patients with CLL and MM, up to two6 cohorts with escalating IV dose levels (and up to 6 parallel IV dose levels+LDSC IL-2) with patients with AML, and up to three cohorts with SC administration with or without LDSC IL-2 (one or two cohort(s) with CLL and MM and one cohort with AML).

In Part 2, 20 patients per disease cohort may be included (approximately 60) and each disease cohort may have a different dose and regimen based on the Part 1 results. Patients who withdraw before the first tumor response assessment for reasons other than toxicity or disease progression may be replaced in consultation between the investigator and sponsor.

### Inclusion Criteria

### MM and CLL Patients are Eligible to be Included in the Trial Only if all of the Following Criteria Apply (for Both Part 1 and Part 2 of the Trial):

(a) Patient must be 18 years of age inclusive or above, at the time of signing the informed consent.
(b) Patients with documented diagnosis of CLL or MM who have failed to respond to or who have relapsed after prior therapy and are not amenable to standard treatments or for whom no standard treatments are available. Patients may have undergone prior cell therapy.

### CLL/Small Lymphocytic Lymphoma (SLL) Patients:

(a) Proven disease by the presence of CD5+CD19+CD23+ clonal B cells in blood, bone marrow and/or lymph nodes.
(b) Patients should meet criteria for requiring therapy (the most recent iwCLL guidelines) and must have measurable disease (measurable lesion >1.5 cm diameter in at least one dimension) and/or lymphocytosis.
(c) Patients must have received at least 2 prior lines of therapy and must have failed at least one line of targeted therapy (ibrutinib or venetoclax or similar) and not be amenable to- or for whom no further standard treatment is available.

### MM Patients:

(a) Documented diagnosis of MM and measurable disease (see Appendix 6; measurable disease is defined as serum monoclonal paraprotein (M-protein) ≥5 g/L or urine M-protein ≥200 mg/24 hours or abnormal free light chain (FLC) ratio with involved FLC >100 mg/L or proven plasmacytoma by biopsy*). * If plasmacytoma is the only measurable parameter, the patient is not allowed to be included in the trial, because of difficult response evaluation.
(b) Documented progression or refractory MM as per the IMWG uniform response criteria following ≥3 prior regimens that include at least one immunomodulatory drug, a proteasome inhibitor, and an anti-CD38 monoclonal antibody in any order.

### Males or Non-Pregnant, Non-Breastfeeding Females Who are:

(a) Surgically sterile (hysterectomy, bilateral oophorectomy or bilateral salpingectomy, vasectomy)
(b) Female of childbearing potential with a negative pregnancy test prior to first dosing and compliant with a highly effective contraceptive regimen (i.e., pregnancy rate of <1% per year: oral contraceptives, intrauterine device (IUD), intrauterine hormone-releasing systems) from signing of the informed consent form (ICF) through 90 days after the last IMP administration. Abstinence is not considered an adequate contraceptive regimen.
(c) Female, postmenopausal defined as continuous amenorrhea for at least 12 consecutive months without an alternative medical cause and/or a serum follicle-stimulating hormone (FSH) measurement of >40 IU/L).
(d) Male, compliant with an effective contraceptive regimen (i.e., use of male condom with female partner and assuring use of an additional highly effective contraceptive method with a failure rate of <1% per year when having sexual intercourse with a woman of childbearing potential who is not currently pregnant following from signing of the ICF through 90 days after the last IMP administration) from signing of the ICF through 90 days after the last IMP administration). Abstinence is not considered an adequate contraceptive regimen.
(e) Male, refraining from donating sperm following from signing of the ICF through 90 days after the last IMP administration.
(f) Predicted life-expectancy of 3 months.
(g) Eastern Cooperative Oncology Group (ECOG) performance status of 0 or 1.
(h) Adequate renal function (creatinine clearance ≥50 mL/min); hepatic function [(total bilirubin ≤1.5 times upper limit of normal (ULN), unless in patients with known Gilbert's syndrome who must have total bilirubin ≤3 times ULN; aspartate aminotransferase (AST) and alanine aminotransferase (ALT) ≤3.0 times ULN] and hematological function (neutrophils ≥1×109/L, unless this is considered due to bone marrow tumor infiltration; platelet count ≥75×109/L, unless due to bone marrow tumor infiltration, in which case it must be ≥50×109/L).
(i) Capable of giving signed and dated informed consent prior to initiation of any trial-related procedure that is not considered Standard of Care which includes compliance with the requirements and restrictions listed in the ICF and in the protocol. This also means that (according to French Public Health Code article L 1121-6, L 1121-8 & L 1126-6) patients should not be (1) deprived of liberty, (2) placed under legal protection or unable to express their consent, (3) hospitalized without consent.

### AML Patients are Eligible to be Included in the Trial Only if all of the Following Criteria Apply (for Both Part 1 and Part 2 of the Trial):

(a) Patient must be 18 years of age inclusive or above, at the time of signing the informed consent.
(b) Patients with documented diagnosis AML and/or MDS (defined using the most recent International Consensus Classification (ICC) guidelines) and who meet the following criteria:
(c) Patients with acute promyelocytic leukemia (APL; AML M3) are excluded.
(d) Patients with MDS should have morphologically confirmed diagnosis as intermediate risk, high risk, or extremely high risk (IPSS-R classification).
(e) Patients with relapsed/refractory AML (defined as relapsed disease, refractory disease, or MRD relapsed disease as per the most recent ELN guidelines).
(f) Patients with MDS must have received at least one prior hypomethylating agent.
(g) Patients are generally not amenable to further standard treatment or for whom no standard treatments are available as per investigator judgement. Patients may have undergone prior autologous stem cell transplant.
(h) Males or non-pregnant, non-breastfeeding females who fullfill any of the following criteria:
(i) Surgically sterile (hysterectomy, bilateral oophorectomy or bilateral salpingectomy, vasectomy)
(j) Female of childbearing potential with a negative pregnancy test prior to first dosing and compliant with a highly effective contraceptive regimen (i.e., pregnancy rate of <1% per year: oral contraceptives, intrauterine device (IUD), intrauterine hormone-releasing systems) from signing of the informed consent form (ICF) through 90 days after the last IMP administration. Abstinence is not considered an adequate contraceptive regimen.
(k) Female, postmenopausal defined as continuous amenorrhea for at least 12 consecutive months without an alternative medical cause and/or a serum follicle-stimulating hormone (FSH) measurement of >40 IU/L).
(l) Male, compliant with an effective contraceptive regimen (i.e., use of male condom with female partner and assuring use of an additional highly effective contraceptive method with a failure rate of <1% per year when having sexual intercourse with a woman of childbearing potential who is not currently pregnant following from signing of the ICF through 90 days after the last IMP administration) from signing of the ICF through 90 days after the last IMP administration). Abstinence is not considered an adequate contraceptive regimen.
(m) Male, refraining from donating sperm following from signing of the ICF through 90 days after the last IMP administration.
(n) Predicted life-expectancy of 3 months.
(o) Eastern Cooperative Oncology Group (ECOG) performance status of 0-2.
(p) Adequate renal function with the following limits unless considered due to leukemia: (Creatinine clearance ≥40 mL/min); hepatic function [(total bilirubin <2 times upper limit of normal (ULN), unless in patients with known Gilbert's syndrome who must have total bilirubin ≤3 times ULN (Patient with leukemic organ involvement as assessed by the study investigator, must have a serum direct bilirubin ≤5.0×ULN); aspartate aminotransferase (AST) and alanine aminotransferase (ALT) ≤3.0 times ULN].
(q) Capable of giving signed and dated informed consent prior to initiation of any trial-related procedure that is not considered Standard of Care which includes compliance with the requirements and restrictions listed in the ICF and in the protocol. This also means that (according to French Public Health Code article L 1121-6, L 1121-8 & L 1126-6) patients should not be (1) deprived of liberty, (2) placed under legal protection or unable to express their consent, (3) hospitalized without consent.

### Exclusion Criteria

MM and CLL Patients are Excluded from the Trial if any of the Following Criteria Apply:
(a) Prior allogeneic bone marrow transplant if the patient still has active acute or chronic graft versus host disease requiring >10 mg prednisone or equivalent corticosteroids.
(b) Concomitant malignancies except carcinoma in situ, basal or squamous cell skin carcinoma. Patients who had no evidence of disease from another primary cancer for 2 or more years are allowed to participate in the trial. Localized non-metastatic prostate cancer, not requiring systemic treatment, and for which no local treatment is planned, is allowed.
(c) Uncontrolled or severe intercurrent medical condition.
(d) Known uncontrolled central nervous system involvement.
(e) Patient has any active-, uncontrolled-, or suspected infection.
(f) A significant history of renal, neurologic, psychiatric, pulmonary, endocrinologic, metabolic, immunologic, cardiovascular, or hepatic disease that in the opinion of the investigator would adversely affect the patient's participation in this trial.
(g) Unstable cardiovascular function defined as: (a) symptomatic ischemia, or (b) uncontrolled clinically significant conduction abnormalities (i.e., ventricular tachycardia on antiarrhythmic agents are excluded; 1st degree atrioventricular block or asymptomatic left anterior fascicular block/right bundle branch block will not be excluded), or (c) congestive heart failure New York Heart Association Class ≥3, or (d) myocardial infarction within 3 months, or (e) QTc >480 msec using Fredericia's QT correction formula.
(h) Previous treatment with radiotherapy, targeted therapy, investigational product, or chemotherapy in the 2 weeks (or 4 weeks for previous T-cell directed immunotherapy if requested by specific regulatory authorities) prior to initial IMP administration.
(i) Previous treatment with an aminobisphosphonate IV (e.g., ibandronate, pamidronate, zoledronate etc) within 4 weeks prior to initial IMP.
(j) Previous treatment of any systemic immunosuppressant within 2 weeks prior to initial IMP administration, with the exception of systemic corticosteroid use up to oral dose of 10 mg prednisolone daily (or equivalent for other steroids).
(k) Previous treatment with live or live attenuated vaccines within 2 weeks prior to initial IMP administration. Other (new) types of vaccines need to be evaluated as to their mode of action.
(l) Previous autologous haematopoietic stem cell transplantation (HSCT) or treatment with Chimeric Antigen Receptor (CAR) T-cell therapy within 6 months prior to initial IMP administration.
(m) Known non-CLL/MM related pre-existing clinically relevant immunodeficiency disorders.
(n) Patients with Richter's transformation are excluded.
(o) Positive serological testing for human immunodeficiency virus (HIV) antibody, hepatitis B surface antigen [HBsAg] and hepatitis B core antibody (anti-HBc) negative, and hepatitis C virus antibody. Patients who are positive for anti-HBc or hepatitis C antibody may be included if they have a negative PCR within 6 weeks prior to initial IMP administration. Those who are PCR positive will be excluded.
(p) Known allergies, hypersensitivity, or intolerance to the excipients of the IMP or IL-2.
(q) Major surgery within 4 weeks of initial IMP administration or planned surgery during the time the patient is expected to participate in the trial.
(r) Known ongoing drug and alcohol abuse in the opinion of the investigator.
   AML Patients are Excluded from the Trial if any of the Following Criteria Apply:
(a) Patient has highly proliferative disease requiring concomitant hydroxurea on trial
(b) Has a white blood cell count >20,000/uL. NOTE: Hydroxyurea is permitted to meet this criterion and should be stopped ≥24 hours before starting treatment on the trial.
(c) Prior allogeneic bone marrow transplant.
(d) Active graft versus host disease requiring >10 mg prednisone or equivalent corticosteroids.
(e) Concomitant malignancies except carcinoma in situ, basal or squamous cell skin carcinoma. Patients who had no evidence of disease from another primary cancer for 2 or more years are allowed to participate in the trial. Localized non-metastatic prostate cancer, not requiring systemic treatment, and for which no local treatment is planned, is allowed.
(f) Uncontrolled or severe intercurrent medical condition as per investigator judgment.
(g) Known central nervous system involvement.
(h) Patient has any active infection or uncontrollable infection requiring systemic treatment (except for mild-low genitourinary system infection and upper respiratory tract infection);
(i) A significant history of renal, neurologic, psychiatric, pulmonary, endocrinologic, metabolic, immunologic, cardiovascular, or hepatic disease that in the opinion of the investigator would adversely affect the patient's participation in this trial.
(j) Unstable cardiovascular function defined as: (a) symptomatic ischemia, or (b) uncontrolled clinically significant conduction abnormalities (i.e., patients with ventricular tachycardia on antiarrhythmic agents are excluded; patients with 1st degree atrioventricular block or asymptomatic left anterior fascicular block/right bundle branch block (left anterior fascicular block/right bundle branch block) will not be excluded), or (c) congestive heart failure New York Heart Association Class ≥3, or (d) myocardial infarction within 3 months, or (e) QTc >480 msec using Fredericia's QT correction formula.
(k) Treatment with radiotherapy, immunotherapy, investigational product, or chemotherapy in the 2 weeks prior to initial IMP administration (with the exception of hydroxurea which must be stopped at least 24 hours prior to initial IMP administration).
(l) Treatment with an aminobisphosphonate IV (e.g., ibandronate, pamidronate, zoledronate etc) within 4 weeks prior to initial IMP.
(m) Treatment of any systemic immunosuppressant within 2 weeks prior to initial IMP administration, with the exception of systemic corticosteroid use up to oral dose of 10 mg prednisolone daily (or equivalent for other steroids).
(n) Treatment with live or live attenuated vaccines within 2 weeks prior to initial IMP administration. Other (new) types of vaccines need to be discussed with the Sponsor as to their mode of action and potential interaction with 1D12-5C8 VAR.
(o) Known non-AML related pre-existing clinically relevant immunodeficiency disorders.
(p) Positive serological testing for human immunodeficiency virus (HIV) antibody, hepatitis B surface antigen [HBsAg] and hepatitis B core antibody (anti-HBc) negative, and hepatitis C virus antibody. Patients who are positive for anti-HBc or hepatitis C antibody may be included if they have a negative PCR within 6 weeks prior to initial IMP administration. Those who are PCR positive will be excluded.
(q) Known allergies, hypersensitivity, or intolerance to the excipients of the IMP or IL-2.
(r) Major surgery within 4 weeks of initial IMP administration or planned surgery during the time the patient is expected to participate in the trial.
(s) Known ongoing drug and alcohol abuse in the opinion of the investigator.

### Duration Patient Participation

The trial duration for an individual patient will be a planned treatment duration of 6 cycles of 28 days (i.e., 24 weeks), screening and follow-up not included. At the request of the treating physician and in consultation with the sponsor, continued access to IMP might be offered beyond the planned treatment duration of 24 weeks for individual patients with ongoing disease control.

### IMP, Dose, Mode of Administration and Dose Regimen

1D12-5C8 VAR is a concentrate for solution for IV or SC administration. For IV administration 1D12-5C8 VAR will be diluted and administered in a 2-hour infusion at Day 1, 8, 11, 15, 18, 22, 25 in the first treatment cycle of 28 days and twice weekly thereafter in Part 1. For SC administration 1D12-5C8 VAR will be injected subcutaneously. The preferred injection site will be the abdomen; the upper arm and thigh are considered as acceptable alternative sites. Injection sites may be rotated for consecutive SC doses, if needed.

Patients in cohort 5 will receive one dose of 1D12-5C8 VAR at Cycle 1 Day 8 and patients in cohorts 7A-11A will receive one dose at Cycle 2 Day 1 by SC administration; all other doses in these cohorts will be administered IV.

In parallel cohorts B, LDSC IL-2 (1 MIU) will be administered once a week (at Day 2, 9, 16 and 23) 24 hours (+/−2 hours time window) after end of infusion (EoI) of 1D12-5C8 VAR during the first two cycles of 1D12-5C8 VAR treatment (Cycle 1 and 2).

Once a RP2D(s) dose and schedule of 1D12-5C8 VAR IV (±LDSC IL-2) administration has been determined, 1D12-5C8 VAR will be evaluated as a SC administration with or without LDSC-IL-2 in separate disease cohorts at a dose that is exposure equivalent to the IV RP2D(s).

In Part 2, 1D12-5C8 VAR will be administered at a dose and regimen as established in Part 1 of the trial.

Dosing of each patient continues until disease progression, unacceptable toxicity, withdrawal of consent or otherwise as specified in the IMP discontinuation criteria with a planned treatment duration of 24 weeks.

In Part 1 of this trial a dose will be reduced after occurrence of any DLT. Treatment should be delayed if any non-hematologic Grade ≥2 toxicities are not resolved to Grade ≤1 or baseline by the time of the next dose (except for AEs that are clearly and incontrovertibly due the underlying disease or extraneous causes).

### Criteria for Evaluation and Analyses

### Safety

(a) Vital signs, physical examination, clinical chemistry, hematology, urinalysis, electrocardiograms (ECG) and documentation of any reported AEs. The severity of AEs will be graded according to the National Cancer Institute's CTCAE version 5.0 (NCI CTCAE) Grading Scale (see the NCI CTCAE web page at http://ctep.cancer.gov for details) with the exception of CRS, which is evaluated and graded according to the ASTCT consensus criteria.

### Pharmacokinetics

(a) Blood samples will be collected to determine 1D12-5C8 VAR concentrations.

### Pharmacodynamics

(a) Blood samples will be collected to evaluate biomarkers of drug response. Biomarkers include binding of 1D12-5C8 VAR to Vγ9Vδ2-T cells and CD1d-expressing tumor cells, Vγ9Vδ2-T cells (activation status and frequency), iNKT cells (activation status and frequency), induction of activation of Vγ9Vδ2-T cells ex vivo when exposed to CD1d (as measured by a functional assay) and general immune monitoring (frequency and activation status of B cells, T cell subsets, natural killer (NK) cells, monocytes, dendritic cells) and induction of cytokines (interleukin (IL)-1β, IL-2, IL-6, IL-8, tumor necrosis factor (TNF)-α, interferon (IFN) γ), granulocyte-macrophage colony-stimulating factor (GM-CSF).
(b) In addition, blood samples (for CLL and AML patients) and bone marrow samples (for MM patients) will be taken for the analysis of CD1d and CD277 expression on tumor cells.

### Immunogenicity

(a) Blood samples will be collected to determine potential development of antidrug antibodies directed against 1D12-5C8 VAR.

### Antitumor Activity of 1D12-5C8 VAR and Disease Evaluation

(a) CLL: Disease status will be evaluated according to the most recent iwCLL guidelines. The assessments consist of (1) peripheral blood clonal B cell frequency (2) peripheral blood MRD assessment (using quantitative highly sensitive flow cytometry) (3) computed tomography (CT)-scan of neck/chest/abdomen/pelvis (4) bone marrow evaluation, including MRD assessment.
(b) MM: Response assessment in accordance with the most recent IMWG criteria. The assessments consist of (1) serum M-protein, urine M-protein, serum FLC and serum/urine immunofixation (2) Bone marrow evaluation (3) Skeletal survey/CT- or MRI scan (bone lesions) (4) Extramedullary plasmacytomas measured by physical examination and CT- or MRI scan.
(c) AML: Disease status will be evaluated according to the most recent ELN criteria. The assessments consist of (1) Peripheral blood analysis for assessment of the frequency of leukemic blasts (2) Bone marrow evaluation (3) MRD assessments.

1. A method of treating a myelodysplastic syndrome in a subject in need thereof comprising administering to the subject:
a multispecific antibody comprising a first binding domain that specifically binds to human CD1d and a second binding domain that specifically binds to a Vγ9Vδ2 T cell receptor.

2. The method of claim 1, further comprising administering a low-dose of IL-2 of less than 2 MIU/day.

3. The method of claim 1, wherein the first antigen binding domain comprises SEQ ID NO: 11 or 12.

4. The method of claim 1, wherein the second antigen binding domain comprises SEQ ID NO: 5, 6, or 7.

5. The method of claim 1, wherein the multispecific antibody comprises an amino acid sequence of SEQ ID NO: 13 or 14.

6. The method of claim 1, wherein the subject has received prior treatment with at least one hypomethylating agent.


SEQUENCE LISTING (14 sequences)

SEQ ID NO: 1 - protein, 5 aa, synthetic construct
NYAMG

SEQ ID NO: 2 - protein, 5 aa, synthetic construct
NYAMS

SEQ ID NO: 3 - protein, 17 aa, synthetic construct
AISWSGGSTSYADSVKG

SEQ ID NO: 4 - protein, 21 aa, synthetic construct
QFSGADYGFGRLGIRGYEYDY

SEQ ID NO: 5 - protein, 130 aa, synthetic construct
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 6 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS

SEQ ID NO: 7 - protein, 130 aa, synthetic construct
EVQLLESGGGLVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTLVTVSS

SEQ ID NO: 8 - protein, 5 aa, synthetic construct
DNVMG

SEQ ID NO: 9 - protein, 16 aa, synthetic construct
TIRTGGSTNYADSVKG

SEQ ID NO: 10 - protein, 11 aa, synthetic construct
TIPVPSTPYDY

SEQ ID NO: 11 - protein, 119 aa, synthetic construct
QVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSS

SEQ ID NO: 12 - protein, 119 aa, synthetic construct
EVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSS

SEQ ID NO: 13 - protein, 254 aa, synthetic construct
QVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSSGGGGSEVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 14 - protein, 254 aa, synthetic construct
EVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSSGGGGSEVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS